(19) United States
(12) Patent Application Publication
(10) Pub. No.: US 2024/0189117 A1
(43) Pub. Date: Jun. 13, 2024

(54) PNEUMATIC SOFT DEXTEROUS HAND FOR PATIENT WITH MISSING FINGER FUNCTIONS AND SOFT ROBOT

(71) Applicant: HUAZHONG UNIVERSITY OF SCIENCE AND TECHNOLOGY, Hubei (CN)

(72) Inventors: Cai-Hua Xiong, Hubei (CN); Wen-Bin Chen, Hubei (CN); Run-zhe Zhang, Hubei (CN); Yu Yan, Hubei (CN); Junjie Cai, Hubei (CN)

(73) Assignee: HUAZHONG UNIVERSITY OF SCIENCE AND TECHNOLOGY, Hubei (CN)

(21) Appl. No.: 18/324,995

(22) Filed: May 28, 2023

(30) Foreign Application Priority Data
Dec. 9, 2022 (CN) 202211584869.6

(51) Int. Cl. A61F 2/58; A61F 2/68

(52) U.S. Cl. CPC A61F 2/586; A61F 2/68; A61F 2002/5038

(57) ABSTRACT

A pneumatic soft dexterous hand for a patient with missing finger functions and a soft robot relate to the technical field of soft robots. The pneumatic soft dexterous hand includes a soft finger, the soft finger includes a rubber casing, a silicone tube, an endoskeleton, and a built-in air bag, and the silicone tube is sleeved in the rubber shell. The silicone tube is formed with a chamber, and the endoskeleton and the built-in airbag are arranged in the silicone tube. The endoskeleton includes a plurality of skeleton modules hinged together, the plurality of the skeleton modules are divided into two groups, and the skeleton modules of the two groups are hinged in an alternating manner in sequence from left to right. The built-in airbag is embedded on the endoskeleton to support the endoskeleton.

### CROSS-REFERENCE TO RELATED APPLICATION

This application claims the priority benefit of China application serial no. 202211584869.6, filed on Dec. 9, 2022. The entirety of the above-mentioned patent application is hereby incorporated by reference herein and made a part of this specification.

### BACKGROUND

### Technical Field

The disclosure relates to the technical field of soft robots, and in particular, relates to a pneumatic soft dexterous hand for a patient with missing finger functions and a soft robot.

### Description of Related Art

Dexterous hands are one of the main characteristics that distinguish humans from other animals. Human hands play a vital role in daily life. Human hands are considerably dexterous and are capable of grasping objects and performing other complex tasks. However, there are a large number of people with hand amputations or congenital hand deformities around worldwide, and hand defects have brought great inconvenience to their lives. For human fingers, in addition to bending and extending, the lateral swing movement also expands the grasping ability of the fingers. Due to its excellent human-computer interaction and friendliness, soft actuators are gradually being used as the basic motion unit of prosthetic hands and thus are used in the design of prosthetic hands. Therefore, the design of a pneumatic soft dexterous hand capable of performing bending, extending, and lateral swing movements that can reconstruct the missing functions of a human hand is of great scientific significance and social value.

A variety of articulated bending actuators are currently available for humanoid soft actuators. However, in addition to the characteristics of active joint bending of finger function, the allowable bending angle of each joint after passive compression has not been widely discussed. Further, the Chinese invention patent CN110497396A discloses a variable stiffness pneumatic soft actuator. The actuator uses several endoskeletons connected in series to increase finger stiffness. However, because the endoskeletons are connected by ball joints, after being subjected to lateral impact, the soft fingers cannot only move laterally at the metacarpophalangeal joints like human fingers, and due to the overall compliance of the actuator, they are forced to undergo uniform lateral bending as a whole. In order to solve the above problems, the freedom of lateral swing of the metacarpophalangeal joints is required to be provided, and high stiffness in the lateral swing direction of the fingers themselves is also required to be ensured. The Chinese invention patent CN114347078A uses a worm gear to achieve the lateral swing movement of fingers. However, its transmission mechanism takes up a large volume, which makes it unsuitable for disabled patients who only have defective fingers.

Pose feedback is an indispensable step in the closed-loop control during the use of prosthetic hands. Due to its excellent precision and high integration, the Bragg grating can be used as the detection unit of soft actuators. The Chinese invention patent CN113940712A uses an optical fiber integrated with a plurality of Bragg gratings to achieve precise control of five fingers of a surgical robot. However, due to the brittleness of the material of the optical fiber itself, the excessively small bending radius of curvature makes an optical fiber sensor unusable. When the sensor is used in the field of prosthetic hands, the near-infinitely small bending radius of curvature between the fingers at the metacarpophalangeal joints prevents the optical fiber from being directly attached to the outer wall of the fingers. Moreover, since the soft actuator may bend in space but the optical fiber feedback bending amount has no directionality, how to determine the direction of the optical fiber bending is also a problem.

### SUMMARY

In response to the above defects or the needs for improvement of the related art, the disclosure provides a pneumatic soft dexterous hand for a patient with missing finger functions and a soft robot designed for overcoming the defects such as incomplete hand functions provided by the currently-available soft prosthetic hands and the defects in the arrangement and detection of optical fiber sensors. During the bending and extending movement of the pneumatic soft dexterous hand, bending ratios of its active and passive movements are consistent with those of a human hand. Further, during the lateral swing movement of the pneumatic soft dexterous hand, an actuator is allowed to have a small size while the folding and unfolding angles of the fingers change linearly with the air pressure. In addition, in the disclosure, based on a single degree of freedom of an endoskeleton, it is further ensured that the soft finger itself does not bend laterally when being applied by an external force.

To achieve the above, according to an aspect of the disclosure, the disclosure provides a pneumatic soft dexterous hand for a patient with missing finger functions. The pneumatic soft dexterous hand includes a soft finger, the soft finger includes a rubber casing, a silicone tube, an endoskeleton, and a built-in air bag, and the silicone tube is sleeved in the rubber shell. The silicone tube is formed with a chamber, and the endoskeleton and the built-in airbag are arranged in the silicone tube.

The endoskeleton includes a plurality of skeleton modules hinged together, the plurality of the skeleton modules are divided into two groups, and the skeleton modules of the two groups are hinged in an alternating manner in sequence from left to right. The built-in airbag is embedded on the endoskeleton to support the endoskeleton.

Further, each skeleton module includes a connecting plate, a limiting shaft, and a rotating shaft, the limiting shaft and one end of the rotating shaft are fixedly connected to the connecting plate, and the connecting plate is further provided with a rotation hole. The other end of the rotating shaft is hinged with the rotation hole of the opposite skeleton module, so that the plurality of the skeleton modules are hinged together to form the endoskeleton.

Further, an optical fiber hole is also provided in a region of the connecting plate adjacent to the rotation hole, and the optical fiber hole is configured to allow an optical fiber to pass through. A plurality of Bragg gratings are integrated on the optical fiber, and the pneumatic soft dexterous hand detects the degree of freedom of each joint through the optical fiber. The optical fiber extends into the silicon tube from an air port at one end of the silicone tube, passes through the plurality of optical fiber holes in sequence, is drawn out from another air port at the end of the silicone tube, and then extends into the adjacent soft finger. The optical fiber is arranged along the skeleton modules in an inner chamber of the silicone tube.

Further, the pneumatic software dexterous hand further includes a finger base, a receiving chamber, and a spring air bag, and the finger base is connected to the soft finger and the receiving chamber. One side of the receiving chamber is movably connected to the finger base, and another end is configured to be connected to a residual limb of a human body. Two opposite ends of the spring air bag are connected to two adjacent finger bases.

Further, one side of the receiving chamber is configured to be attached to the residual limb of the human body, the other side is provided with an arc-shaped dovetail groove. The arc-shaped dovetail groove is matched with the finger base to form a movable connection, so as to ensure that a finger is able to deflect and move along the arc-shaped dovetail groove.

Further, hardness of the rubber casing is greater than that of the silicone tube, an opening of one end of the rubber casing is configured to allow the silicone tube to pass through, and the other end is pointed and closed. Hollow gaps are formed in regions of the rubber casing corresponding to a metacarpophalangeal joint, a proximal joint, and a distal joint.

Further, gaps bottom lines of the gaps corresponding to the metacarpophalangeal joint, the proximal joint, and the distal joint and adjacent to a palm side are set differently, and lengths of the gap bottom lines determine a proportional coefficient corresponding to joint bending.

Further, the rubber casing is provided with gap protrusions on side walls of the gaps corresponding to the distal joint and the proximal joint in an axial direction of the rubber casing.

Further, the spring air bag includes a first connecting block, a second connecting block, a variable-diameter spring, and a non-stretchable plastic film, and two ends of the variable-diameter spring are connected to the first connecting block and the second connecting block. The plastic film is formed in a cylindrical shape, and two ends of the plastic film are connected to the first connecting block and the second connecting block. The variable-diameter spring is located in the plastic film, and a cross-sectional diameter of the variable-diameter spring gradually decreases from the middle to both ends along an axis of the variable-diameter spring itself.

According to another aspect of the disclosure, the disclosure further provides a soft robot, and a soft hand of the soft robot is the abovementioned pneumatic soft dexterous hand for a patient with missing finger functions.

In general, compared with the related art through the above technical solutions conceived by the disclosure, the pneumatic soft dexterous hand for a patient with missing finger functions and the soft robot exhibit the following beneficial effects.
1. In the disclosure, by arranging the internally-hinged endoskeleton and the built-in airbag, the lateral swing direction, moving, bending, and twisting of the soft finger of the dexterous hand are restricted during the grasping process. In this way, it is ensured that the dexterous hand exhibits the flexibility of a soft actuator, and the movement precision is also improved.
2. In the disclosure, the air bag embedded with the variable-diameter spring is used as the actuator for the lateral swing movement of the dexterous hand. In this way, it is ensured that the movement function is provided while the actuator is allowed to be small and light while. The folding and unfolding angles of the dexterous hand are thus accordingly controlled.
3. Through the use of the hollow rubber casing to achieve joint bending, it is ensured that the achievable bending angles and bending ratios of the fingers of the dexterous hand are consistent with those of the human hand no matter whether the soft fingers are bent under active pressure or are passively deformed under pressure during the bending and extending movement.
4. An optical fiber integrated with plural Bragg gratings is used to measure the posture of the dexterous hand, and according to the single degree of freedom of the endoskeleton and the finger base, the definiteness of the grating bending direction is ensured. In this way, the problem of failure of the optical fiber sensor due to the excessively small curvature radius of the finger gap is prevented from occurring.

### BRIEF DESCRIPTION OF THE DRAWINGS

FIG. 1 is a schematic view of installation of a pneumatic soft dexterous hand for a patient with missing finger functions provided by the disclosure.

FIG. 2 is a partially exploded schematic view of the pneumatic soft dexterous hand for a patient with missing finger functions in FIG. 1.

(a) and (b) in FIG. 3 are respectively a schematic view and a cross-sectional view of the pneumatic soft dexterous hand for a patient with missing finger functions in FIG. 1.

FIG. 4 is a schematic view of a degree of freedom of movement of a rubber casing of a soft finger in FIG. 3.

FIG. 5 is a schematic view of installation of an endoskeleton, a built-in airbag, and an optical fiber of the soft finger in FIG. 3.

FIG. 6 is a schematic view of a skeleton module of the endoskeleton in FIG. 5.

FIG. 7 is a schematic view of the built-in air bag in FIG. 5.

FIG. 8 is a schematic view of the pneumatic soft dexterous hand for a patient with missing finger functions provided by the disclosure from another viewing angle.

### DESCRIPTION OF THE EMBODIMENTS

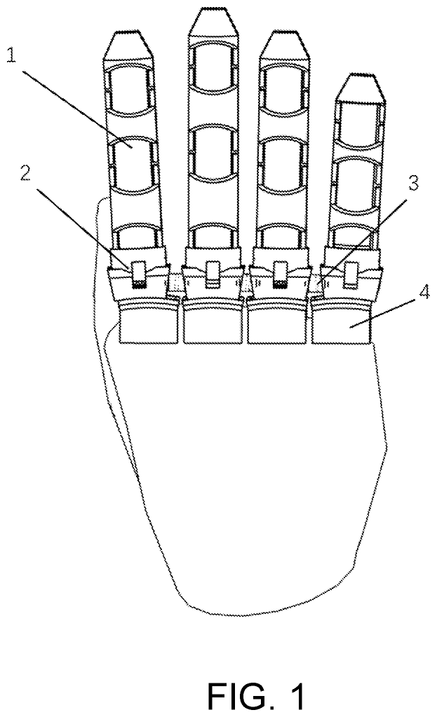

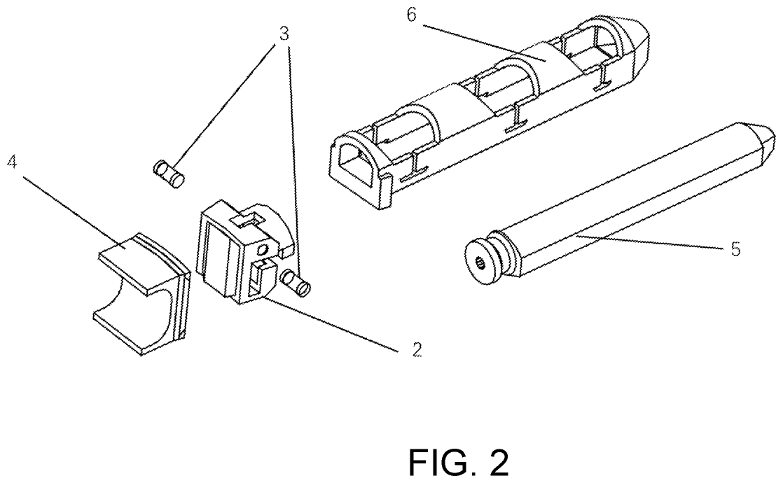

In order to make the objectives, technical solutions, and advantages of the disclosure clearer and more comprehensible, the disclosure is further described in detail with reference to the drawings and embodiments. It should be understood that the specific embodiments described herein serve to explain the disclosure merely and are not used to limit the disclosure. In addition, the technical features involved in the various embodiments of the disclosure described below can be combined with each other as long as the technical features do not conflict with each other. With reference to FIG. 1 and FIG. 2, the disclosure provides a pneumatic soft dexterous

hand for a patient with missing finger functions. The pneumatic software dexterous hand includes a finger 1, a finger base 2, a receiving chamber 4, and a spring air bag 3, and the finger base 2 is connected to the soft finger 1 and the receiving chamber 4. One side of the receiving chamber 4 is movably connected to the finger base 2, and another end is configured to be connected to a residual limb of a human body. Adjacent finger bases 2 are connected through the spring air bag 3. Two opposite ends of the spring air bag 3 are connected to two adjacent finger bases 2. In this embodiment, the number of the soft fingers 1, the number of the finger bases 2, and the number of the receiving chambers 4 are the same. A single soft finger 1 is the basic unit, and a patient with missing fingers can flexibly choose the number of fingers to wear according to his/her own conditions.

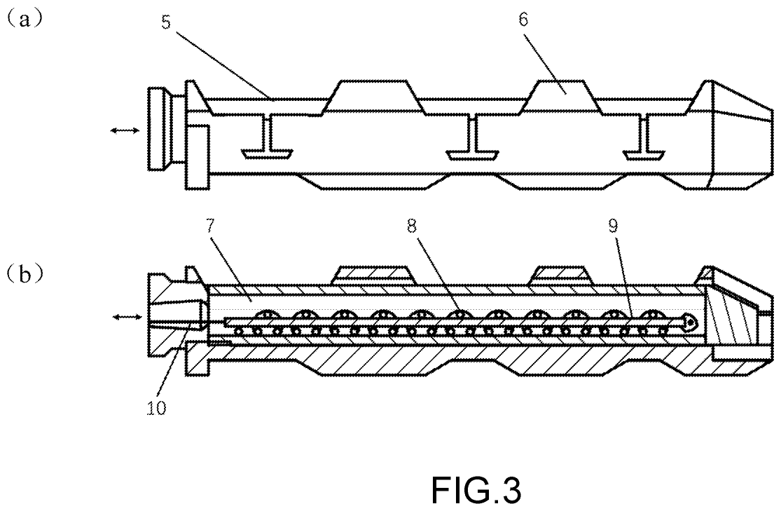

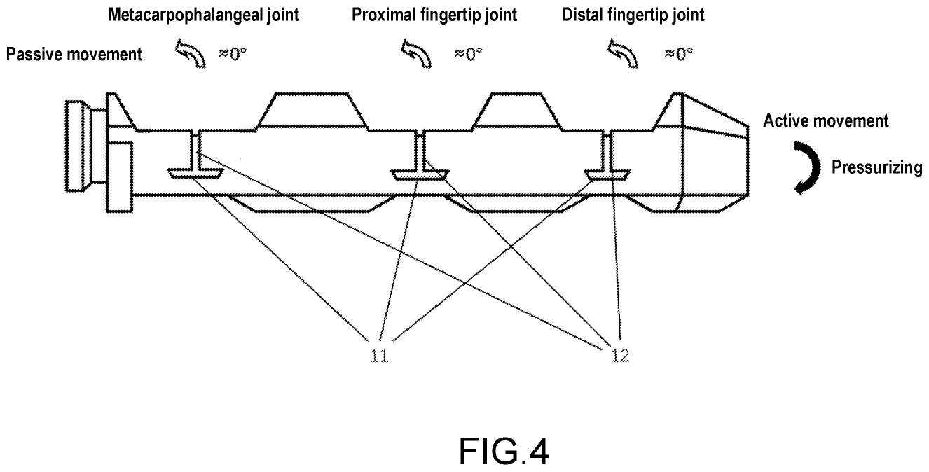

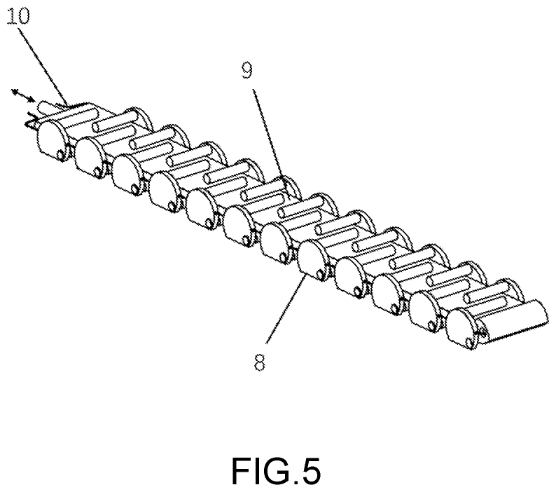

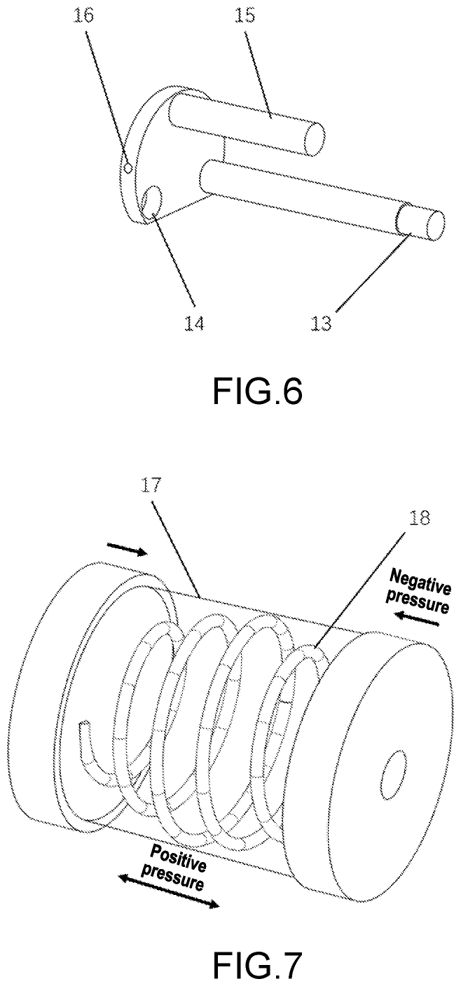

With reference to FIG. 3, FIG. 4, FIG. 5, FIG. 6, and FIG. 7, the soft finger 1 includes a rubber casing 6, a silicone tube 5, an endoskeleton 8, and a built-in air bag 9. An opening of one end of the rubber casing is configured to allow the silicone tube 5 to pass through, and the other end is pointed and closed. Hollow gaps are formed in regions of the rubber casing 6 corresponding to a metacarpophalangeal joint, a proximal joint, and a distal joint. Since the stiffness at the gaps is low, the soft finger 1 may bend at the gaps after the silicone tube 5 is pressurized. Lengths of gap bottom lines of the gaps adjacent to a palm side determine a proportionality coefficient of bending of the joints. The lengths of the gap bottom lines of the gaps corresponding to the three joints are set differently, so that the finger joints can be bent approximately in a specific ratio.

Hardness of the rubber casing 6 is greater than that of the silicone tube 5. The silicone tube 5 is sleeved in the rubber casing 6, and after being squeezed and elongated, the silicone tube 5 may squeeze a tip end of the rubber casing 6 to generate a moment towards the palm side and cause the soft finger 1 to bend inwards. Stiffness at gap bottom sides 11 is low, so that the rubber casing 6 only undergoes bending deformation at the gap bottom sides 11 after being compressed. Further, under the same bending moment, since the gap bottom sides 11 have the same amount of bending per unit length, adjustment of the lengths of the gap bottom sides 11 can make the bending ratio among the finger joints approximately 3:2. As such, the movement function of the human hand may be reconstructed during the active movement.

The opening of one end of the rubber casing is configured to allow the silicone tube 5 to pass through, and the other end is pointed and closed. Since the stiffness at the gaps is low, the gap bottom sides 11 at the gaps of the soft finger 1 may bend inwards after the silicone tube 5 is pressurized. The lengths of gap bottom sides 11 determine the proportionality coefficient of the bending angles of the joints. The gap bottom sides 11 correspond to the three joints and are set differently, so that each single finger joint can be bent approximately in a specific ratio.

The rubber casing 6 is provided with gap protrusions 12 on side walls of the gaps corresponding to the distal joint and the proximal joint in an axial direction of the rubber casing 12. When the soft finger 1 is deformed towards the back of the hand due to an external pressure, the gap protrusions 12 may limit the movement of the joints where the fingers passively move after bending and touching and may limit the bending range of the joints in the direction of the back of the hand. As shown in FIG. 4, the range of movement of a fingertip joint and the metacarpophalangeal joint is approximately equal to 0°, which ensures the normal movement of the human hand during the passive movement of the soft finger 1 under pressure.

Glass fiber is wound around the outer periphery of the silicone tube 5, and a chamber 7 is formed inside the silicone tube 5. The endoskeleton 8 and the built-in airbag 9 are arranged in the chamber 7. Herein, the glass fiber is configured to limit the radial expansion of the silicone tube 5, so that the silicone tube 5 only stretches axially during the pressurization process.

The endoskeleton 8 includes a plurality of skeleton modules hinged together. The plurality of the skeleton modules are divided into two groups, and the skeleton modules of the two groups are hinged in an alternating manner in sequence from left to right. In this way, the degree of freedom of finger bending and extending is ensured, and lateral bending or twisting is limited. The built-in airbag 9 is embedded on the endoskeleton 8 to support the endoskeleton 8. In this way, it is ensured that the endoskeleton 8 can be inserted into the soft finger 1 at one time, and the manufacturing process is thus simplified.

Each skeleton module includes a connecting plate, a limiting shaft 15, and a rotating shaft 13. The limiting shaft 15 and one end of the rotating shaft 13 are fixedly connected to the connecting plate, and the connecting plate is further provided with a rotation hole 14. The central axis of the rotation hole 14, the central axis of the limiting shaft 15, and the central axis of the rotating shaft 13 all pass through the same triangle vertex. Herein, the other end of the rotating shaft 13 is hinged with the rotation hole 14 of the opposite skeleton module, so that the plurality of the skeleton modules are hinged together to form the endoskeleton 8. An optical fiber hole 16 is also provided in a region of the connecting plate adjacent to the rotation hole 14, and the optical fiber hole 16 is configured to allow an optical fiber 10 to pass through. In this embodiment, the central axis of the optical fiber hole 16 is perpendicular to the central axis of the rotation hole 14.

The limiting shafts 15 of two opposite skeleton modules are arranged in an alternating manner, and the built-in airbag 9 is located between the limiting shafts 15 and the rotating shafts 13. Since the rotating shaft 13 needs to be matched with the rotation hole 14, under this restriction, the endoskeleton 8 can only move in a plane perpendicular to the axis of the rotating shaft 13. Considering the large number of parts of the endoskeleton 8 and the inability to maintain a stable state by itself, the non-stretchable built-in airbag 9 made of plastic may ensure the vertical state of the endoskeleton 8 after pressurization. Therefore, due to the support provided by the built-in airbag 9 to the endoskeleton 8, the endoskeleton 8 can be inserted into the chamber 7 of the silicone tube 5 at one time and fixed in position during the manufacturing process. The limiting shaft 15 is configured provide the same effect as the rotating shaft 13 to limit the built-in airbag 9 inside the endoskeleton 8. The combined use of the silicone tube 5 and the endoskeleton 8 provides the flexibility of the fingers of the dexterous hand and also ensures that the soft finger 1 has the bending resistance of the joints of the human hand in the side swing direction.

The finger bases 2 are connected by the spring air bags 3, so that the dexterous hand can perform lateral swinging movement. Herein, one side of the receiving chamber 4 is configured to be attached to the residual limb of the human body, and the other side is provided with an arc-shaped dovetail groove. The arc-shaped dovetail groove is matched with the finger base 2, so as to ensure that a finger is able to deflect and move along the arc-shaped dovetail groove.

The spring air bag includes a first connecting block, a second connecting block, a variable-diameter spring 18, and a non-stretchable plastic film 17. Two ends of the variable-diameter spring 18 are connected to the first connecting block and the second connecting block. The plastic film 17 is formed in a cylindrical shape, and two ends thereof are connected to the first connecting block and the second connecting block. The variable-diameter spring 18 is located in the plastic film 17. In the initial state, the inside of the spring air bag 3 is provided with a negative pressure, the variable-diameter spring 18 is compressed, and the fingers of the dexterous hand are folded together. In the working state, the inside of the spring air bag 3 is provided with a positive pressure, the variable-diameter spring 18 returns, and the fingers of the dexterous hand laterally swing. The elongation of the air bag embedded with the spring changes linearly with the change of pressure intensity, so the amplitude of lateral swing of the fingers may thus be accordingly adjusted. A cross-sectional diameter of the variable-diameter spring 18 gradually decreases from the middle to both ends along an axis of the variable-diameter spring 18 itself. Therefore, when the variable-diameter spring 18 is compressed, each layer of spring of the variable-diameter spring 18 can overlap each other, so that the spring length is limited in a thin plane.

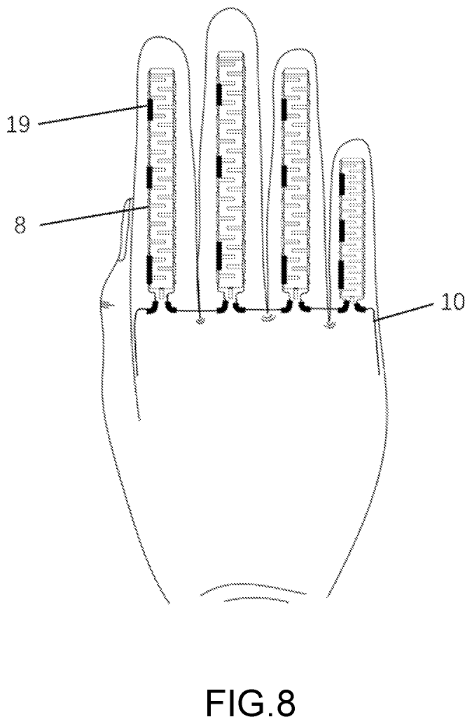

With reference to FIG. 8, the dexterous hand uses a single optical fiber integrated with a plurality of Bragg gratings 19 to collect the degree of freedom of each joint. Since the feature of inflexibility provided by the optical fiber 10 conflicts with the compressible deformation feature of the soft finger 1, direct insertion the optical fiber 10 into the silicone tube 5 may cause the optical fiber 10 to break during the deformation process. Further, since directionality cannot be determined from the data fed back by the Bragg gratings 19 when the Bragg gratings 19 feed back the bending, the optical fiber 10 is embedded in the optical fiber hole 16 of each component unit in the endoskeleton 8. The optical fiber 10 extends into the silicon tube 5 from an air port at one end of the silicone tube 5. The components of the endoskeleton 8 have the fiber holes 16 for insertion of the optical fiber. The optical fiber is arranged in the inner chamber of the silicone tube 5 along the endoskeleton 8, basically in a U-shaped arrangement, is drawn out from another air port at the end of the silicone tube 5, and then extends from the finger base 2 into the adjacent soft finger 1. The Bragg gratings 19 are arranged at the corresponding joints of the dexterous hand, the nearly 180° bending is therefore split into two bending angles, and normal operation is thus ensured.

The disclosure further provides a soft robot, and a soft hand of the soft robot is the abovementioned pneumatic soft dexterous hand for a patient with missing finger functions.

A person having ordinary skill in the art should be able to easily understand that the above description is only preferred embodiments of the disclosure and is not intended to limit the disclosure. Any modifications, equivalent replacements, and modifications made without departing from the spirit and principles of the disclosure should fall within the protection scope of the disclosure.

What is claimed is:

1. A pneumatic soft dexterous hand for a patient with missing finger functions, wherein
the pneumatic soft dexterous hand comprises soft fingers, each of the soft fingers comprises a rubber casing, a silicone tube, an endoskeleton, and a built-in air bag, the silicone tube is sleeved in the rubber shell, the silicone tube is formed with a chamber, and the endoskeleton and the built-in airbag are arranged in the silicone tube,
the endoskeleton comprises a plurality of skeleton modules hinged together, the plurality of the skeleton modules are divided into two groups, the skeleton modules of the two groups are hinged in an alternating manner in sequence from left to right, and the built-in airbag is embedded on the endoskeleton to support the endoskeleton.

2. The pneumatic soft dexterous hand for a patient with missing finger functions according to claim 1, wherein the skeleton module comprises a connecting plate, a limiting shaft, and a rotating shaft, the limiting shaft and one end of the rotating shaft are fixedly connected to the connecting plate, the connecting plate is further provided with a rotation hole, and the other end of the rotating shaft is hinged with a rotation hole of an opposite skeleton module of the skeleton modules, so that the plurality of the skeleton modules are hinged together to form the endoskeleton.

3. The pneumatic soft dexterous hand for a patient with missing finger functions according to claim 2, wherein optical fiber holes are also provided in a region of the connecting plate adjacent to the rotation hole, the optical fiber hole is configured to allow an optical fiber to pass through, a plurality of Bragg gratings are integrated on the optical fiber, degree of freedom of each joint of the pneumatic soft dexterous hand is detected through the optical fiber, the optical fiber extends into the silicon tube from an air port at one end of the silicone tube, passes through the optical fiber holes in sequence, is drawn out from another air port at the end of the silicone tube, and then extends into an adjacent soft finger of the soft fingers, and the optical fiber is arranged along the skeleton modules in the chamber of the silicone tube.

4. The pneumatic soft dexterous hand for a patient with missing finger functions according to claim 1, wherein the pneumatic software dexterous hand further comprises finger bases, receiving chambers, and spring air bags, the finger bases are connected to the soft fingers and the receiving chambers, one side of the receiving chamber is movably connected to the finger base, and the other end of the receiving chamber is configured to be connected to a residual limb of a human body, and two opposite ends of each of the spring air bags are connected to two adjacent finger bases of the finger bases.

5. The pneumatic soft dexterous hand for a patient with missing finger functions according to claim 4, wherein the one side of the receiving chamber is configured to be attached to the residual limb of the human body, the other side of the receiving chamber is provided with an arc-shaped dovetail groove, and the arc-shaped dovetail groove is matched with the finger base to form a movable connection, so as to ensure that the soft finger is able to deflect and move along the arc-shaped dovetail groove.

6. The pneumatic soft dexterous hand for a patient with missing finger functions according to claim 4, wherein hardness of the rubber casing is greater than hardness of the silicone tube, an opening of one end of the rubber casing is configured to allow the silicone tube to pass through, the other end of the rubber casing is pointed and closed, and hollow gaps are formed in regions of the rubber casing corresponding to a metacarpophalangeal joint, a proximal joint, and a distal joint.

7. The pneumatic soft dexterous hand for a patient with missing finger functions according to claim 6, wherein gaps bottom lines of the hollow gaps corresponding to the metacarpophalangeal joint, the proximal joint, and the distal joint and adjacent to a palm side are set differently, and lengths of the gap bottom lines determine a proportional coefficient corresponding to joint bending.

8. The pneumatic soft dexterous hand for a patient with missing finger functions according to claim 6, wherein the rubber casing is provided with gap protrusions on side walls of the gaps corresponding to the distal joint and the proximal joint in an axial direction of the rubber casing.

9. The pneumatic soft dexterous hand for a patient with missing finger functions according to claim 4, wherein the spring air bag comprises a first connecting block, a second connecting block, a variable-diameter spring, and a non-stretchable plastic film, two ends of the variable-diameter spring are connected to the first connecting block and the second connecting block, the plastic film is formed in a cylindrical shape, two ends of the plastic film are connected to the first connecting block and the second connecting block, the variable-diameter spring is located in the plastic film, and a cross-sectional diameter of the variable-diameter spring gradually decreases from the middle to both ends along an axis of the variable-diameter spring itself.

10. A soft robot, wherein a soft hand of the soft robot is the pneumatic soft dexterous hand for a patient with missing finger functions according to claim 1.

11. The soft robot according to claim 10, wherein the skeleton module comprises a connecting plate, a limiting shaft, and a rotating shaft, the limiting shaft and one end of the rotating shaft are fixedly connected to the connecting plate, the connecting plate is further provided with a rotation hole, and the other end of the rotating shaft is hinged with a rotation hole of an opposite skeleton module of the skeleton modules, so that the plurality of the skeleton modules are hinged together to form the endoskeleton.

12. The soft robot according to claim 11, wherein optical fiber holes are also provided in a region of the connecting plate adjacent to the rotation hole, the optical fiber hole is configured to allow an optical fiber to pass through, a plurality of Bragg gratings are integrated on the optical fiber, degree of freedom of each joint of the pneumatic soft dexterous hand is detected through the optical fiber, the optical fiber extends into the silicon tube from an air port at one end of the silicone tube, passes through the optical fiber holes in sequence, is drawn out from another air port at the end of the silicone tube, and then extends into an adjacent soft finger of the soft fingers, and the optical fiber is arranged along the skeleton modules in the chamber of the silicone tube.

13. The soft robot according to claim 10, wherein the pneumatic software dexterous hand further comprises finger bases, receiving chambers, and spring air bags, the finger bases are connected to the soft fingers and the receiving chambers, one side of the receiving chamber is movably connected to the finger base, and the other end of the receiving chamber is configured to be connected to a residual limb of a human body, and two opposite ends of each of the spring air bags are connected to two adjacent finger bases of the finger bases.

14. The soft robot according to claim 13, wherein the one side of the receiving chamber is configured to be attached to the residual limb of the human body, the other side of the receiving chamber is provided with an arc-shaped dovetail groove, and the arc-shaped dovetail groove is matched with the finger base to form a movable connection, so as to ensure that the soft finger is able to deflect and move along the arc-shaped dovetail groove.

15. The soft robot according to claim 13, wherein hardness of the rubber casing is greater than hardness of the silicone tube, an opening of one end of the rubber casing is configured to allow the silicone tube to pass through, the other end of the rubber casing is pointed and closed, and hollow gaps are formed in regions of the rubber casing corresponding to a metacarpophalangeal joint, a proximal joint, and a distal joint.

16. The soft robot according to claim 15, wherein gaps bottom lines of the hollow gaps corresponding to the metacarpophalangeal joint, the proximal joint, and the distal joint and adjacent to a palm side are set differently, and lengths of the gap bottom lines determine a proportional coefficient corresponding to joint bending.

17. The soft robot according to claim 15, wherein the rubber casing is provided with gap protrusions on side walls of the gaps corresponding to the distal joint and the proximal joint in an axial direction of the rubber casing.

18. The soft robot according to claim 13, wherein the spring air bag comprises a first connecting block, a second connecting block, a variable-diameter spring, and a non-stretchable plastic film, two ends of the variable-diameter spring are connected to the first connecting block and the second connecting block, the plastic film is formed in a cylindrical shape, two ends of the plastic film are connected to the first connecting block and the second connecting block, the variable-diameter spring is located in the plastic film, and a cross-sectional diameter of the variable-diameter spring gradually decreases from the middle to both ends along an axis of the variable-diameter spring itself.

19. The pneumatic soft dexterous hand for a patient with missing finger functions according to claim 2, wherein the pneumatic software dexterous hand further comprises finger bases, receiving chambers, and spring air bags, the finger bases are connected to the soft fingers and the receiving chambers, one side of the receiving chamber is movably connected to the finger base, and the other end of the receiving chamber is configured to be connected to a residual limb of a human body, and two opposite ends of each of the spring air bags are connected to two adjacent finger bases of the finger bases.

20. The pneumatic soft dexterous hand for a patient with missing finger functions according to claim 3, wherein the pneumatic software dexterous hand further comprises finger bases, receiving chambers, and spring air bags, the finger bases are connected to the soft fingers and the receiving chambers, one side of the receiving chamber is movably connected to the finger base, and the other end of the receiving chamber is configured to be connected to a residual limb of a human body, and two opposite ends of each of the spring air bags are connected to two adjacent finger bases of the finger bases.
